(19) United States
(12) Patent Application Publication
(10) Pub. No.: US 2003/0195390 A1
(43) Pub. Date: Oct. 16, 2003

(54) Digital laryngoscope

(76) Inventor: Martin Panczel Graumann, Encino, CA (US)

Correspondence Address: Martin Panczel Graumann, PO Box 261012, Encino, CA, 91426-1012, US

(21) Appl. No.: 10/070,850

(22) PCT Filed: Mar. 11, 2002
(86) PCT No.: PCT/US02/07170

(51) Int. Cl. A61B 1/267

(52) U.S. Cl. 600/188

(57) ABSTRACT

The Digital Laryngoscope is a Layngoscope design that incorporates the latest optical and digital technology for a reliable and consistent method of visualization and exposure of anatomical structures required for Endotracheal intubation.

It consists of a Blade unit with a ‘reverse’ curvature or concave distal end outfitted with a light system. The Blade's proximal end forms the Blade to Handle mounting system, based on a ‘slide-mount’ mechanism. The Handle unit's proximal end has Handle to Blade receiver ‘slide-mount’ mechanism, a curved metal Tube that houses at its distal end an Optical image sensor and its wiring. The Tube is mounted to the Handle via attachments to the receiver ‘slide-mount’ and the Handle itself. The Handle is ergonomically shaped and angled. The Handle serves as the housing unit for the Digital color processor and its wiring connecting it to the Tube unit's Lens system, as well as the lid-shaped molded Battery power supply.

The distal end of the Handle is outfitted with a mounting, connector/receiver that mounts/connects to a Radio-frequency Transitter that transmits the image via remote wireless mode onto one or several display monitor screens fitted with a radio-frequency receiver, and it does so simultaneously.

The Handle can accommodate connection of a detachable small and compact color LCD Monitor supported on an adjustable, swivel support. The displays as well as the LCD Monitor display the image of the visual field in full color and picture quality resolution.

### BACKGROUND OF THE INVENTION

References Cited

| | | | | |
|---|---|---|---|---|
| 5827178 | January | 1998 | Berrall | 600/185,188 |
| 3884222 | May | 1975 | Moore | |
| 4491865 | January | 1985 | Danna et al. | |
| 4651202 | March | 1987 | Arakawa | |
| 4677471 | June | 1987 | Takamura et al. | |
| 4736734 | April | 1988 | Matsuura et al. | |
| 4877016 | October | 1989 | Kantor et al. | |
| 4878485 | November | 1989 | Adair | 128/6 |
| 4901708 | February | 1990 | Lee | 128/11 |
| 4918521 | April | 1990 | Yabe et al. | |
| 4989586 | February | 1991 | Furukawa | |
| 5363838 | November | 1994 | George | |
| 5363839 | November | 1994 | Lankford | |
| 5408992 | April | 1995 | Hamlin | |
| 5494483 | February | 1996 | Adair | |
| 5527261 | June | 1996 | Monroe et al. | |
| 4086919 | May | 1978 | Bullard | |
| 5178131 | January | 1993 | Upsher | |
| 5263472 | November | 1993 | Ough | |
| 5800344 | October | 1998 | Wood et al. | 600/188.185 |

The Laryngoscope is a specialized medical instrument used for instrumentation of the patients airways to facilitate exposure, visualization and endo-tracheal intubation of the trachea.

It is a widely used instrument by multiple medical specialties and medical personnel World wide. In its most specialized purpose serves as the most relied upon and used Anesthesiology instrument. In addition it finds its use in all hospitals, operating rooms, intensive care units, emergency and trauma rooms, life-flights, fire stations, and paramedics gear.

The procedure of laryngoscopy in which it is used is performed to establish an airway, and often is as a life-saving procedure. Therefore reliance upon for predictable performance under difficult circumstances and variable conditions associated with patient to patient anatomical variations, places a rather high demand upon its performance and reliabiabiability.

With this perspective in mind there has been an ongoing search for continued improvements to perfect its performance at every level possible.

The most often encountered failure in it performance is its inability to allow exposure and visualization of the Laryngeal anatomy such as Pharynx and Vocal Cords to pass an endo-tracheal tube into the Trachea and securing the airway of the patient.

This is most often due to excessive soft tissue in heavy patients, or abnormal Maxillo-facial structures. Under these circumstances ‘blind’ attempts to intubate may cause a cascade of associated complications when unsuccessful. These complications are in and of themselves present a threat to life.

The present day advances in improving the Laryngoscope has focused on replacing the necessity to use direct visualization of the anatomic structures by the utilization of available technology, i.e. fiberoptics, fiberoptic video scopes adapted from other medical uses.

These devices provide an indirect and more maneuverable option, by replacing the human eye as a direct visual instrument that must see into the mouth through a limited opening and around often none displaceable structures.

The use of available technology to facilitate and transmit the image of the Pharyngeal and Tracheal anatomy to outside the mouth where the operator performing the procedure is more conveniently able to visualize has been the focus of most resent innovations and invention.

The frequency of failure at first attempts to intubate is directly proportional with operator training, experience, patients weight and variations of the maxillofacial anatomy.

The Laryngoscope's weakness lies in its Blade design. It presents limitations due to its shape design often fail when anatomic variations are encountered.

The procedure of Laryngoscopy requires that the Blade be inserted into the mouth, displacing the tongue, base of the tongue and reaching under the Pharyngeal structure and lifting the Epiglottis that covers, conceals and protects the Tracheal opening, and Vocal cords.

When displacing, lifting and exposing the tracheal opening is not accomplished at first attempt, all subsequent attempts necessitate more force and manipulation of the Blade which causes undue collateral damage to teeth, soft tissues with its associated bleeding, swelling and distortion of anatomy.

When failure necessitates ‘blind’ intubation its success rate is rather low and accidental Esophageal intubation carries its life threaten complications such as gastric reflux, aspiration pneumonitis and increasing morbidity and mortality.

The ultimate of all life threatening complication when a Laryngoscope fails is the inability to establish an airway, i.e. intubate or ventilate the patients Lungs. As to date the number one cause of operating room deaths are caused by “inability to establish an airway‘,leading to cardiac arrest and often brain injury.

Therefore the Laryngoscope is a critically important instrument that must be used and relied upon for performance, under life threatening conditions that places an ever increasing demand for technical improvements for reliability and predictability of performance.

### SUMMARY OF THE INVENTION

The present invention provides a Laryngoscope with structural and technical design characteristics that defines its advantages and improvement of its performance and reliability during its use.

The Laryngoscope is comprised of a Blade unit, a Handle unit, a Tubing unit, an Optical image sensor unit, Handle to Blade Coupler-mount unit, a Digital color processor unit, a Battery power supply unit, on or more remote wireless Display color monitor units fitted with a Radio-frequency receiver and an LCD Monitor display unit.

The Blade is an ergonomically shaped by design with a concave distal portion that facilitates displacement of the most obstructive structure encountered, and facilitates exposure of the subepiglotic anatomy, namely the vocal cords and the tracheal opening.

Thus aiding the performance of layngoscopic endo-tracheal intubation process whether performed under direct or indirect visual control.

The Blade is fitted with an Infrared Light Emitting Diode mounted to the distal end for illuminating the anatomical structures of the visual field, and its proximal end forms the first part of the Coupler-mount system for mounting it to the Handle unit a slide-mount mechanism.

The Handle holds the Tube unit, that is contoured to fit the Blade's bending curvature, independently behind the Blade unit, extending near its distal end. The Tube unit houses an Optical image sensor unit, wired and sealed, connecting it to the Handle and its Digital Color Processor.

The Handle's proximal end contains the second part of the Coupler-mount unit for Blade attachment, as well as the Digital Color Processor electronic circuits. The Digital processor receives its input from the Optical image sensor via wiring and sends it by wire to a connecting mount that connects the Handle to a Radio-Frequency Transmitter (RFT) unit.

The Handle holds a form-molded Battery unit that serves as a power supply for the Digital Processor electronics as well as for the RFT unit. The Battery forms part of the Handle and it is an integral part thereof.

The RFT receives its input from the Digital Processor unit and transmits it to one or multiple Remote Wireless Display color monitor screens fitted with a Radio-frequency Receiver (RFC) unit. The image of the visual field of the anatomic structures are thus visualized and displayed in full color and picture quality resolution.

The RFT may be replaced with a small and compact color LCD Monitor that connects to the Handle's distal end connector. The LCD color display monitor mounted to an adjustable support mount for viewing the visual field displayed in full color and picture type resolution.

Additional details of its features are described in the detailed description of the invention.

### BRIEF DESCRIPTION OF THE DRAWINGS

Drawing 1; is a full side view of the invention unit with the numbered elements as per FIGS. 1-13 depicted.

FIG. 1 is the Blade and its Light system FIG. 5 protruding through the Blade's flange. It is mounted to the Handle unit FIG. 6 via Coupler-mount system FIG. 3 and Lock-knob FIG. 4.

FIG. 2 is the contoured Tube housing the Optical image sensor and wiring mounted to the Coupler-mount FIG. 3 and Handle FIG. 6.

FIG. 6 is the Handle, holding the Coupler-mount FIG. 3 and Tube FIG. 2, as well as the form-molded Battery FIG. 7.

FIG. 8 is the connector-mount to FIG. 9 and support swivel FIG. 10 to LCD monitor FIG. 11. Drawing 2; is a side view of the invention unit FIGS. 2-11 with the Blade unit FIG. 2 removed.

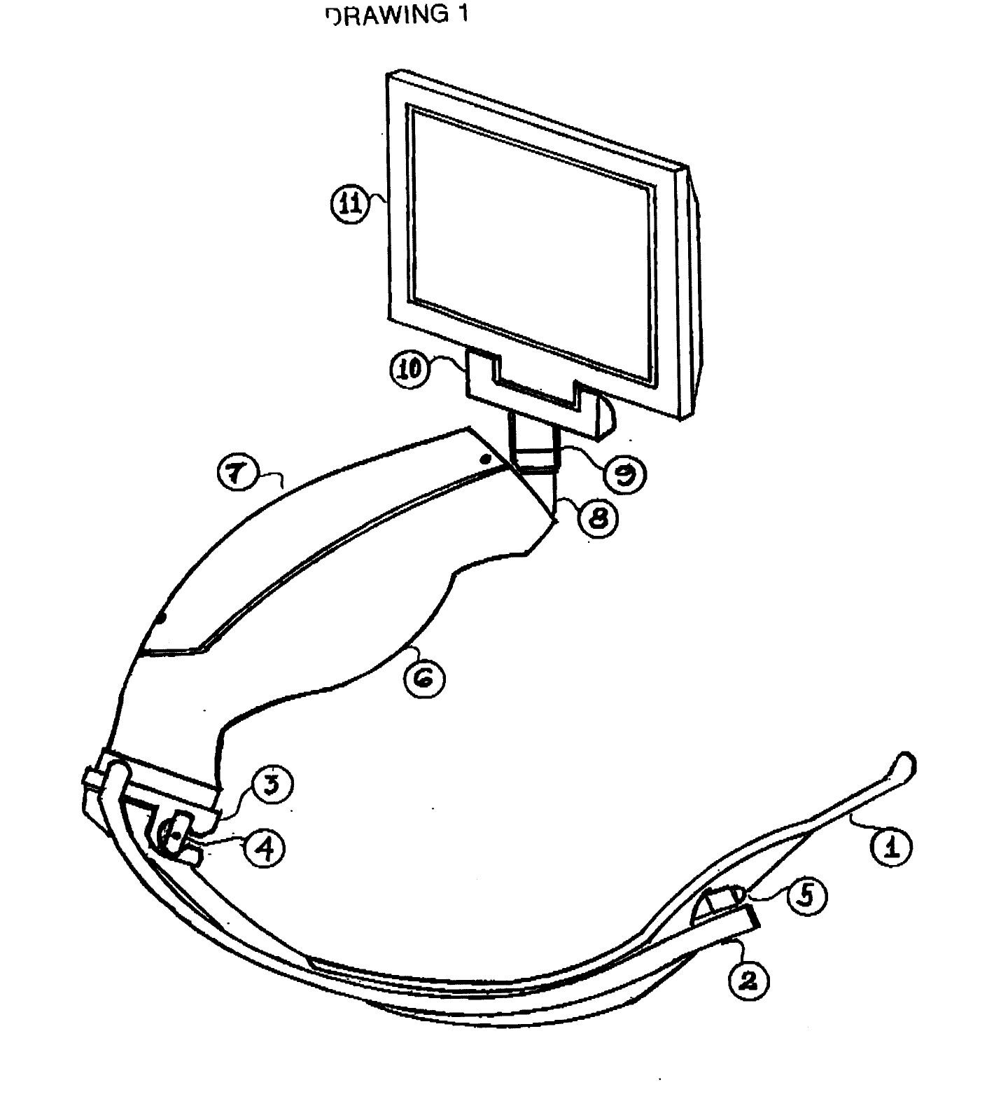

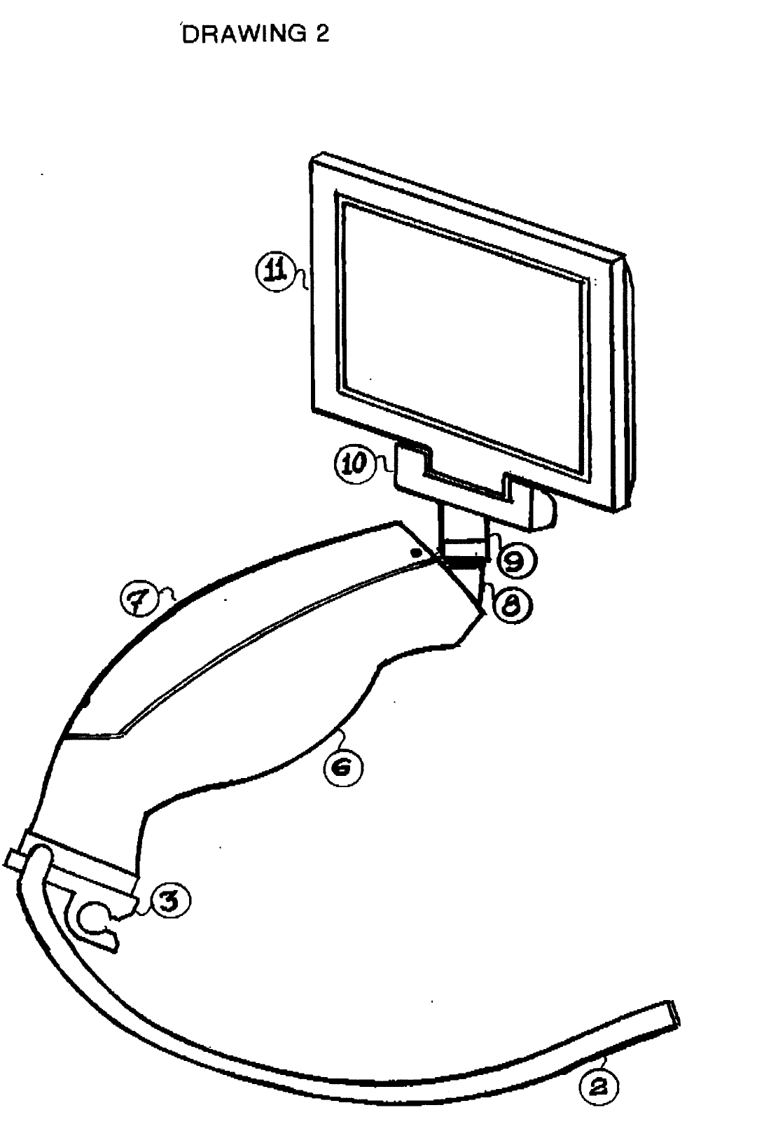

Drawing 3; a 3-dimensional side view of the Blade FIG. 1 detached from Coupler-mount system FIG. 3. Its distal end with ‘reveresed-curvature’/concave surface contour, with its Light system FIG. 5, and proximal end ‘male’ coupler-mount’ and Lock-knob FIG. 4.

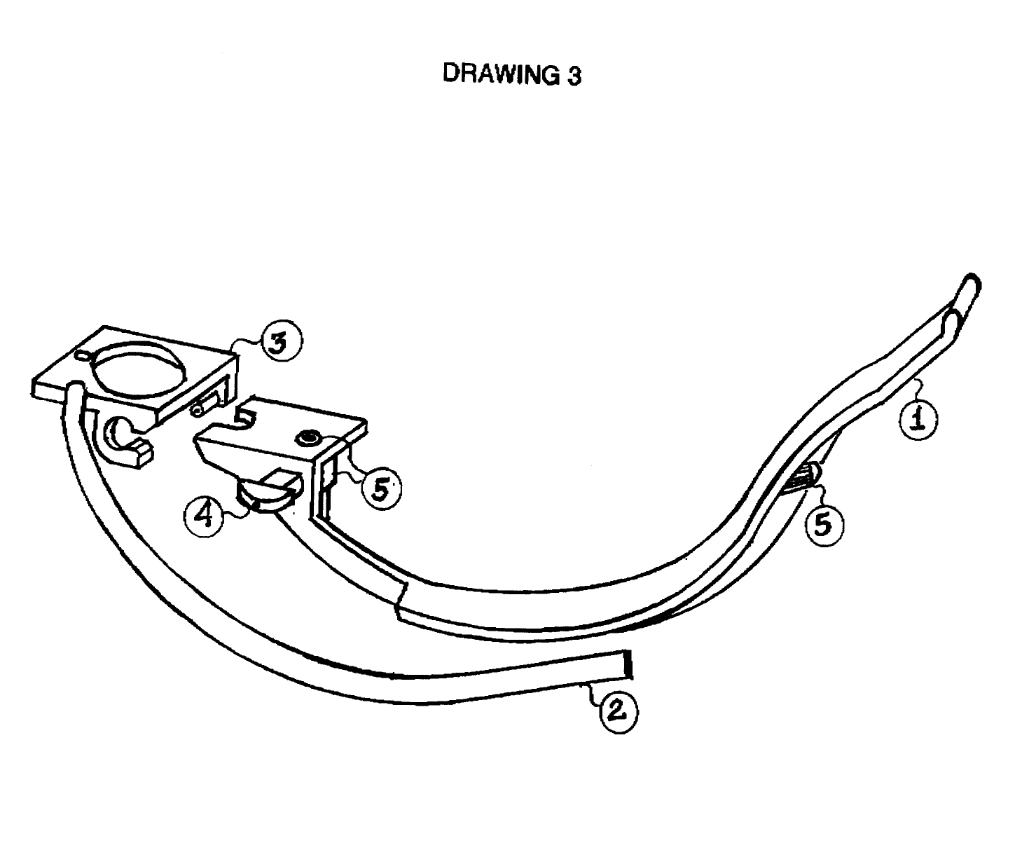

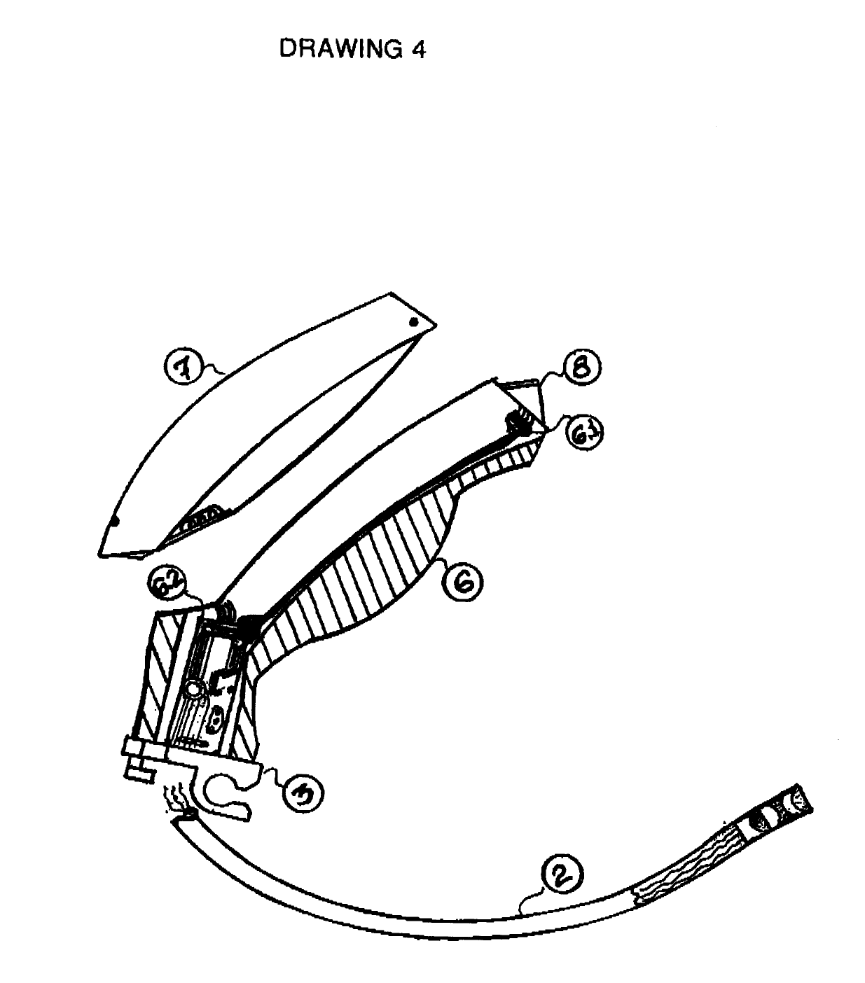

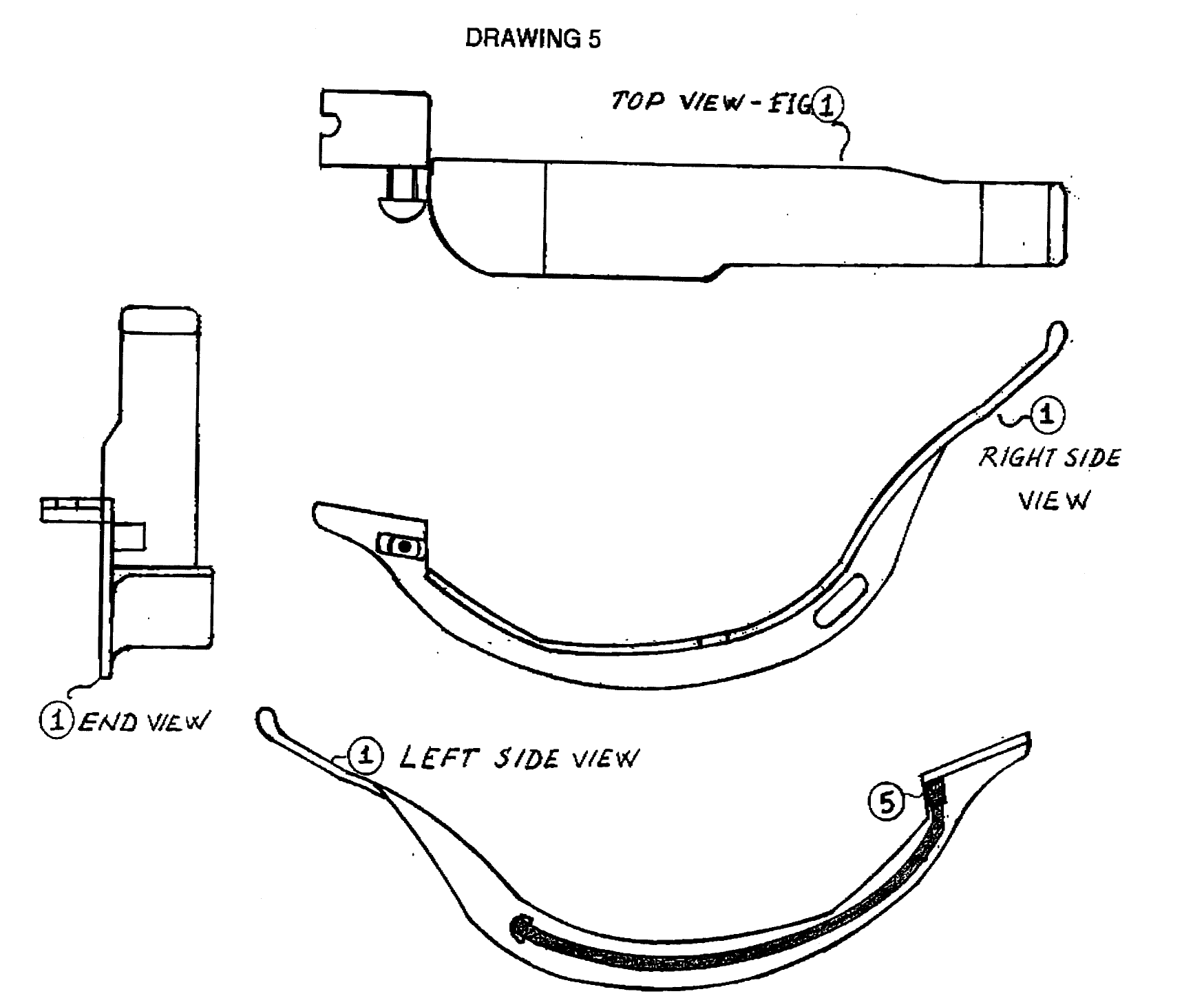

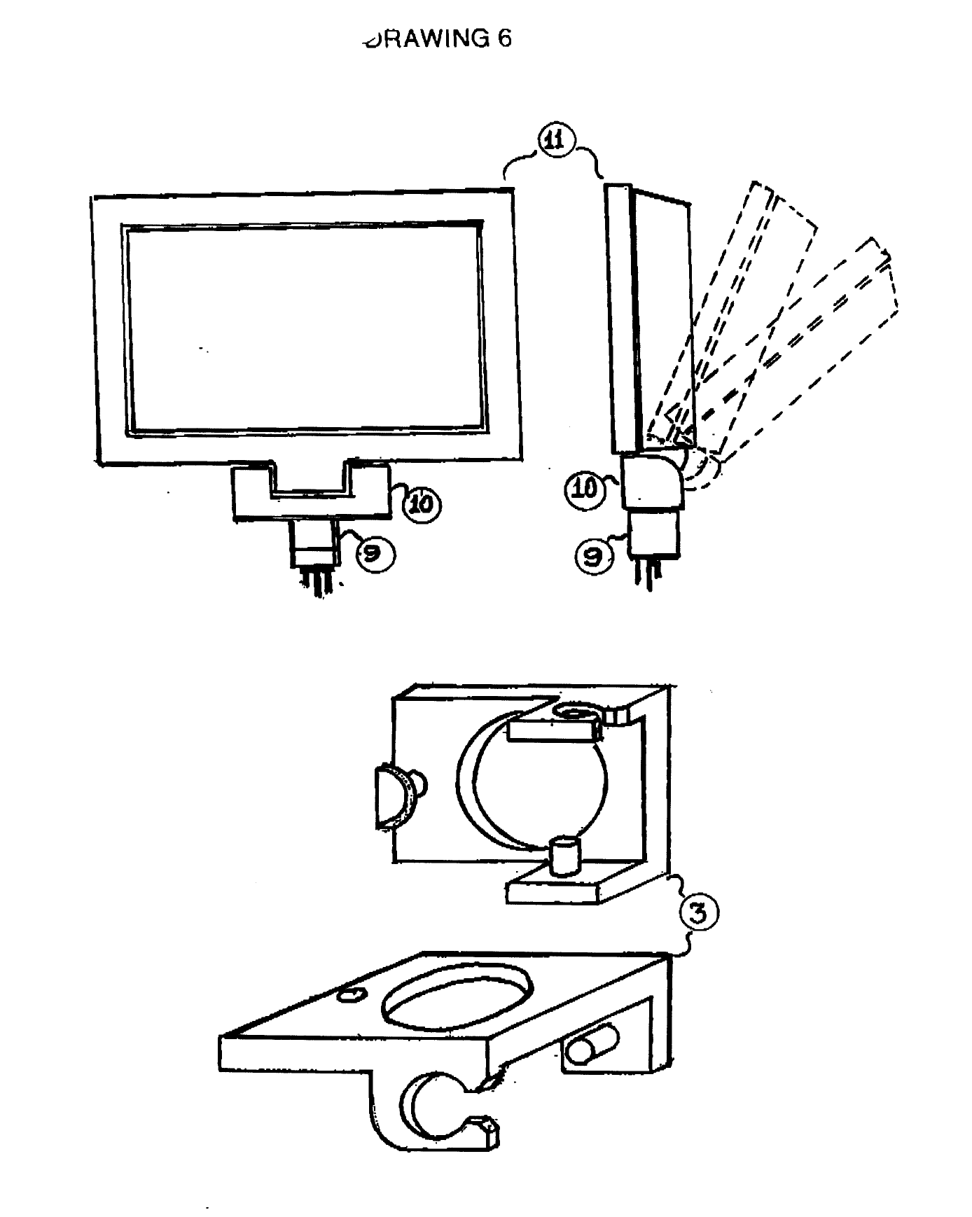

a 3-dimensional view of Tube unit FIG. 2 and ‘female’ Coupler-mount unit FIG. 3 without Handle FIG. 6

Drawing 4, a side view of FIG. 2 distal end a cut-away showing Optical image sensor system, distal end of Tube FIG. 2 and its proximal end cut showing wiring at proximal end and Coupler-mount FIG. 3 with Hanlde FIG. 6 section view exposing digital circuit board/chip FIG. 6.2 wiring, connectors FIG. 6.1 and Battery FIG. 7 removed.

Drawing 5; a Left/Right side views of Blade FIG. 1 with Light FIG. 5 wiring and connector housed in sealed tube

an end view FIG. 1, top view of FIG. 1 with ‘male’ coupler-mount proximal end with Lock-knob FIG. 4.

Drawing 6; 3-dimensional views of the ‘female’ Coupler-mount element subassembly FIG. 3 and LCD monitor FIG. 11 front and side wievs with connector FIG. 9 and mount support FIG. 10.

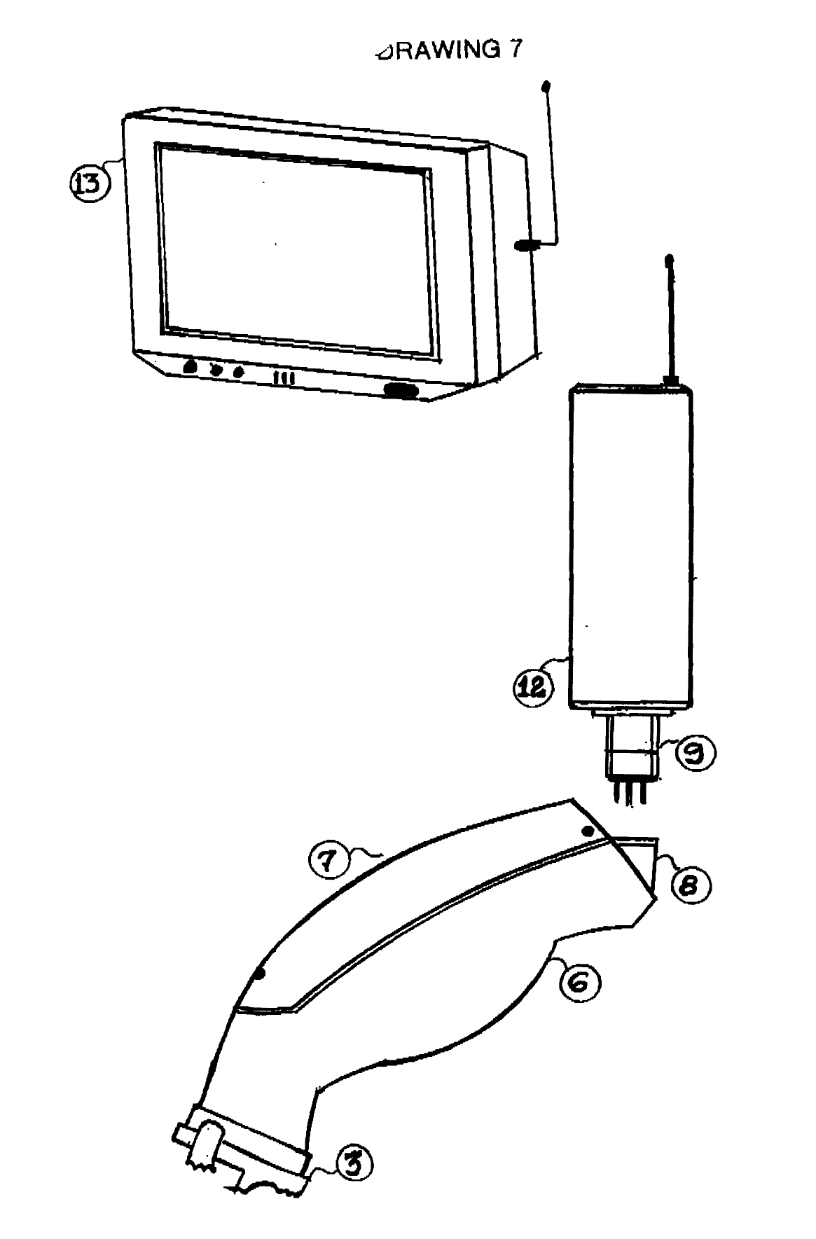

Drawing 7; side view of FIG. 3 cut, Handle FIG. 6 and Battery FIG. 7 with FIG. 8 and FIG. 9 connector/holder of Radio-Frequency Transmitter (RFT) FIG. 12 and remote receiver monitor FIG. 13 fitted with radio-frequency receiver.

### DETAILED DESCRIPTION OF THE INVENTION

The Laryngoscope described in the invention is featured in FIGS. 1-13. The Laryngoscope preferred embodiments are: a Blade unit FIG. 1, a Handle unit FIG. 6, a Tubing unit FIG. 2, an Optical unit housed in the Tube unit FIG. 2, a Handle to Blade, Coupler-Mount unit FIG. 3, a Digital Color Processor unit FIG. 6.2 housed in the Handle unit, a Battery/Power supply unit FIG. 7 housed in the Handle, a Radio-Frequency Transmitter (RFT) unit FIG. 12, a single or multiple Wireless Remote Display Monitor screen(s) unit FIG. 13 with Radio-Frequency Receivers (RFC) units and an LCD Color Display Monitor unit FIG. 11.

The Blade FIG. 1 is ergonomically shaped by design to accommodate and conform to anatomical structures upon which it is designed to act. At its distal portion it incorporates a concave/reverse curvature surface positioned to facilitate maximum displacement when applied to the most obstructive part, base of the tongue, of the anatomical structures it must displace to aid in exposure of subepiglottic structures, namely the vocal cords and trachea to facilitate and aid in the procedure of endotracheal intubation under an indirect or direct and continuous visual control technique.

The Blade is fitted with an Inrared Light Emitting Diode(LED) or Light Bulb FIG. 5, and mounted to the Blade's flange near the distal end. This provides illumination of the anatomical structures of the visual field. The LED FIG. 5 is wired into a hermetically sealed tubing affixed to the outer flange of the Blade, and reaches a contact-connector FIG. 5 on the Blade's proximal end, that forms the Blade to Handle Coupler-mount unit.

The Blade's proximal end of its flange is thinned to allow manipulation and pitching the blade during its use, without the risk of tooth damage.

The Blade's proximal end forms a part of the Coupler-Mount unit FIG. 3, that enables assembly to the Handle unit, and is fitted with a Lock-knob FIG. 4 for securing it to Handle. The Coupler-mount second part FIG. 3 is affixed to the Handle FIG. 6 forming a slide-mount mechanism for Blade mounting. This slide-mount provides for fast and easy Blade changing without disruption to the Optical unit, Tube housing FIG. 2.

The Handle unit holds the Tube unit FIG. 2, that is contoured and designed to fit the Blade's bending radius, independently following its curvature behind the Blade FIG. 1, extending near its distal end.

The Tube unit FIG. 2 distal end houses the Optical unit and its wiring that connects it to the Digital Color Processor FIG. 6.2.

The Optical image sensor unit is composed of a lens system that collects the light image input from its visual field, projects it onto a sensor with 0-0.7 Lux sensitivity, that converts it into signals transmitted to the Digital Processor. The Tube unit FIG. 2 provides a hermetically sealed system and its proximal end is mounted to the Handle FIG. 6 and Coupler-mount FIG. 3.

Thus, the Handle/Tube unit is completely independent of the Blade FIG. 1. The Digital color processor FIG. 6.2 housed in the Handle FIG. 6, receives its input by direct wire connection with the Optical image sensor unit of FIG. 2 and sends it by direct wire connection to a connector mount FIG. 6.1&FIG. 8 that receives connector FIG. 9 with its mounting of RFT FIG. 12.

The Handle FIG. 6 is ergonomically contoured, and mounted at such an angle relative to the Blade's averaged axis, as to facilitate maximum force transfer to the blades concave curvature and distal tip and facilitate displacement of the encountered soft tissues namely the base of the tongue.

The Handle FIG. 6 accommodates a form-molded Battery FIG. 7 that serves as power supply for the electronics of the Digital processor FIG. 6.2, the LED FIG. 5 and RFT FIG. 12. It is form-molded to conform to and become an integral part of the Handle. It is rechargeable and removable.

The RFT FIG. 12 receives its input from the Digital color processor unit FIG. 6.2 and transmits it by predetermined radio-frequency waves settings, via remote wireless mode to one or multiple Display monitor screens outfitted with a Radio-Frequency Receiver(RFC), that are located in the same and/or other locations.

The displayed image may be view by one or many individuals in full color and picture quality resolution. This adds a valuable feature to the invention by allowing utilization for training, teaching or supervising purposes in addition to utilization for the instrumentation of the airways and the performance of Laryngoscopic endo-tracheal intubations.

The Handle FIG. 6 may be individually fitted with a small 3-4 inch LCD Monitor FIG. 11. The LCD Monitor with its separate disposable battery/power supply housed behind its back panel is connected to the Handle via FIGS. 8-9.

The LCD mounted on an adjustable swivel and rotation support FIG. 10, that allows screen adjustment for viewing the image display in color and picture quality resolution. One of the LCD monitor feature is that it is fully removable for safety and storage.

The invention herein described provides alternative and improved methods for the instrumentation and application of direct or indirect visual application for the procedure of Laryngoscopic endo-tracheal intubation, as well as an instrument guided examination, inta-operative instrumental visualization of the Pharyngeal anatomy during short laser procedures of the Vocal cords as well as the proximal end of the Tracheal opening.

What I claim as my invention is;

1. A laryngoscope comprising a blade, a handle, a coupler-mount means for mounting the blade to handle, a tube
housing an optical means mounted into its distal end for collecting and transmitting visual image signals of the visual field,
a digital processor means that processes the image signal, a radio-frequency transmitter,
a radio-frequency receiver outfitted display monitor(s), a battery power supply form-molded into the
handle and an LCD monitor display.

2. A laryngoscope as claimed in claim 1, used for laryngoscopic instrumentation of patients airways,
for diagnostic examination,
orogharyngeal surgical procedures and direct or indirect visual means of endo-tracheal intubation of the trachea.

3. A laryngoscope blade as claimed in claim 1, for displacing the patients tongue and exposure of the tracheal
opening during the process of endo-tracheal intubation.

4. A laryngoscope blade as claimed in claim 1, comprising a convex proximal surface contour and a distal portion concave surface contour as an essential element of its working surface.

5. A laryngoscope blade as claimed in claim 4, comprising a light means mounted near the distal end as a light source
means of illuminating the visual field.

6. A laryngoscope blade as claimed in claim 5, wherein the light source is a low energy, infrared light emitting diode (LED).

7. A laryngoscope blade as claimed in claim 4, wherein the blade is press formed, with a bending radius to form a surface contour and a flange.

8. A laryngoscope blade as claimed in claim 7, wherein the proximal end is fitted to form a means to couple-mount to the handle.

9. A laryngoscope blade as claimed in 8, wherein the proximal end mounting means is supported on the flange.

10. A laryngoscope blade as claimed in 9, wherein the proximal end flange is formed and thinned to provide for
maneuvering and pitching the blade without damage to teeth during the procedure.

11. A laryngoscope blade as claimed in 8, wherein the proximal end is fitted with a lock-knob means.

12. A laryngoscope blade as claimed in claim 8, wherein the blade to handle mounting is by slide-mount means.

13. A laryngoscope handle as claimed in claim 1, wherein the proximal end provides a coupler-mount means for blade to
handle mounting.

14. A laryngoscope handle as claimed in claim 1, wherein the proximal end provides a tube unit means mounted to handle
and coupler-mount.

15. A laryngoscope handle as claimed in claim 1, wherein handle houses a digital processor means mounted in the proximal end.

16. A laryngoscope handle as claimed in claim 1, wherein the handle is contoured to provide an ergonomic means for force
distribution to the blades distal end during the laryngoscopic procedure.

17. A laryngoscope handle as claimed in claim 1, wherein the handle is mounted to blade at an angle relative to the
blade's averaged longitutinal axis.

18. A laryngoscope handle as claimed in claim 1, wherein the handle holds a form-molded battery means as power supply,
that is molded to form an integral part of the handle.

19. A laryngoscope handle as claimed in claim 1, wherein the handle provides means to connect-mount a detachable radio-frequency transmitter.

20. A laryngoscope handle as claimed in claim 1, wherein the handle is fitted to provide means to connect-mount a detachable LCD monitor.

21. A laryngoscope as claimed in claim 1, wherein tube unit houses an optical image sensor means to collect light
image signals from the visual field.

22. An optical image sensor as claimed in claim 21, wherein the optical image sensor and a digital color
processor means are connected by wire.

23. An optical image sensor as claimed in claim 21, wherein it is hermetically sealed into a contoured tube
unit that conforms to the blade curvature and not part of the blade.

24. An optical image sensor as claimed in claim 23, wherein operates at 0-0.7 Lux sensitivity.

25. A laryngoscope as claimed in claim 1, wherein a radio-frequency transmitter connected to handle's distal end by a connector mount.

26. A radio-frequency transmitter as claimed in claim 26 wherein it receives its input from a digital color processor means and transmits by preset radio frequency waves to one or multiple color display monitors in the
same or multiple other locations.

27. The display monitors as claimed in claim 26, wherein the display monitors are fitted with a radio-frequency receiver.

28. The display monitors as claimed in claim 27, wherein the display monitors receive the visual image from the visual field by remote and wireless means.

29. Display monitors as claimed in claim 28, wherein the displayed image is in full color, and picture quality resolution.

30. A laryngoscope handle as claimed in claim 1, wherein an LCD monitor display is connected to its distal end for display of visual field.

31. An LCD monitor as claimed in claim 30, wherein it is a small, compact display, mounted by means of
a support connector mount that allows axial and swivel viewing adjustment.

32. An LCD monitor as claimed in claim 31, wherein display of the visual field input is in color, and picture
quality resolution, and comprises a battery power supply mounted under its back panel.
